(19) United States
(12) Patent Application Publication
(10) Pub. No.: US 2015/0342650 A1
(43) Pub. Date: Dec. 3, 2015

(54) LOCKABLE INTRAMEDULLARY NAIL COMPRISING GUIDE WIRE PASSAGES

(71) Applicant: Cornelius BOBBERT, Wartenberg (DE)

(72) Inventor: Ulrich SCHREIBER, Munich (DE)

(21) Appl. No.: 14/653,405

(22) PCT Filed: Dec. 18, 2013
(86) PCT No.: PCT/EP2013/077207

§ 371 Date: Jun. 18, 2015

(30) Foreign Application Priority Data
Dec. 18, 2012 (DE) 10 2012 024 688.9

(51) Int. Cl. A61B 17/72

(52) U.S. Cl. CPC A61B 17/7241; A61B 17/725

(57) ABSTRACT

The present invention relates to a medullary nail (1) for the insertion into a long bone, with a shaft (5) with a longitudinal axis, wherein the shaft possesses at least one passage opening (8) for inserting at least one locking means, in particular one locking screw (3) or one locking pin, into an inside of the medullary nail (1) and/or for guiding through the medullary nail (1); wherein the medullary nail (1) possesses at least one uptake means (6) for taking up at least one section (3a) of the locking means; wherein the at least one uptake means (6) is disposed inside the shaft or inside the passage opening (8) while it is in at least one of its spatial axes movable or alignable, particularly rotatable or pivotable, in relation to the longitudinal axis of the shaft (5); wherein the medullary nail (1) possesses at least one means, particularly an arresting means (9) and/or an embodiment of locking means and/or uptake means (6), for preventing the alignability of the at least one uptake means (6) in relation to the longitudinal axis and/or to a transverse axis perpendicular thereto; and wherein the at least one uptake means (6) possesses at least one guide wire opening (15) passing therethrough for taking up a guide wire.

The invention relates to a medullary nail and a device for fixing the medullary nail in a long bone according to the pre-characterising portion of the independent patent claim.

Medullary nails and particularly lockable medullary nails are known auxiliary means for the treatment of long bone fractures, which are placed inside the medullary cavity of the damaged bone in order to mechanically bridge the bone lesion. In the so-called locking medullary nails, locking screws, locking pins or generally locking means, which are inserted to bridge and/or connect bone and locking nail, particularly across or inside the bone and the locking nail or sections thereof, serve to secure the connection between bone and locking nail against shifting, particularly of the locking nail, in the direction of the bone axis as well as against rotation of the medullary nail around its longitudinal axis.

It is the problem of the present invention to provide another medullary nail, particularly a locking medullary nail (which in the following is also referred to as medullary nail).

The problem according to the invention is solved by a medullary nail or a locking medullary nail with the features of the independent claim.

According to this, a medullary nail for insertion into a long bone is proposed according to the invention, which possesses a shaft with a longitudinal axis. The shaft possesses at least one passage opening for insertion of at least one locking means, particularly one locking screw or one locking pin, into the inside of the medullary nail and/or for guiding through the medullary bone.

The medullary nail possesses at least one uptake means for taking up at least one section of the locking means.

The at least one uptake means is in at least one of its spatial axes movable or alignable in relation to the longitudinal axis of the shaft. It is particularly rotatably or pivotably disposed on the medullary nail, inside the shaft and/or inside the passage opening.

The medullary nail possesses at least one means, particularly an arresting means and/or an embodiment of a locking means and/or uptake means, for preventing the alignability of the at least one uptake means in relation to the longitudinal axis and/or to a transverse axis perpendicular thereto.

The at least one uptake means possesses at least one guide wire opening passing therethrough for the taking up, guiding through or surrounding of a guide wire. The at least one guide wire opening can be described synonymously as guide wire passage or as guide wire guide.

In certain embodiments according to the invention, a guide wire is understood to be a wire (or a structure, which is also suitable and/or used in practice for that purpose), which is inserted before the insertion of an orthopaedic or surgical instrument and/or implant into the tissue, for example into the bone. In this, the guide wire is provided to insert an instrument or an implant into that tissue along, surrounding, enclosing, connected with and/or guided by the guide wire in a later, second procedure step, into which tissue the guide wire was already inserted in an earlier, first procedure step. In these embodiments, the guide wire serves as a path finder, enabler or lead structure for the instrument or the implant by preparing and/or simplifying its correctly localised insertion in the same tissue.

In all the previous and following embodiments, the use of the expression “may be” and “may have” etc. is to be understood to be synonymous to “preferably is” and “preferably has”, respectively, and is intended to describe exemplary embodiments.

Exemplary embodiments of the present invention may possess one or several of the features mentioned below, individually or in any combination.

Additional exemplary embodiments of the invention are the subject matter of the dependent claims.

The at least one guide wire opening of the medullary nail according to the invention differs in some exemplary embodiments from a passage opening of the uptake means, through which the locking means passes through the uptake means.

In certain embodiments according to the invention, the guide wire opening has a diameter, which is too small for guiding a locking screw therethrough.

In some embodiments according to the invention, the medullary nail is an arthrodesis nail, for example for the stiffening of joints.

In some exemplary embodiments of the medullary nail according to the invention, the at least one guide wire opening passes in some of its sections through the passage opening of the uptake means, in other sections it does not.

In certain exemplary embodiments of the medullary nail according to the invention, the at least one guide wire opening possesses a diameter or a width in a range between 1 and 5 mm, preferably between 2 and 4 mm, preferably of 3 mm. In certain exemplary embodiments according to the invention the guide wire opening possesses a maximum diameter or width of 3 mm. Advantageously, this allows for a sufficient thickness and solidity of the structures surrounding the guide wire opening.

In some exemplary embodiments of the medullary nail according to the invention, the at least one guide wire opening passes not only through the one or the several uptake means, but it can also pass through optionally provisioned intermediary bodies, which can optionally be provisioned between adjacent uptake means. According openings or passages can also be provisioned herein.

Embodiments according to the invention may have one or several of the advantages mentioned herein. These include the ones mentioned below.

The provision of a guide wire opening advantageously permits inserting the medullary nail according to the invention along a guide wire passing through the medullary nail. In this, the medullary nail is only driven into the bone, when its future position and its insertion path are probed and set as well as—where applicable—checked, for example by x-ray, using the previously inserted guide wire. In this way, before the insertion of the medullary nail, the surgeon can be more certain in assuming that the medullary nail will come to rest in the desired way.

Another advantage of guide wire openings can be that by means of a guide wire inserted therein, the position of the uptake means during the more or less forcible driving in of the nail into the bone is reliably retained. The guide wire prevents an accidental rotation of the one or the several uptake means, which could occur due to vibrations during the insertion etc. Because of the position of the uptake means retained by the guide wire with respect to the medullary nail or inside the medullary nail, it can be ensured that openings of the individual uptake means, through which the locking pin(s) are put in after the insertion of the medullary nail, still align (or align for the first time) after the insertion of the medullary nail into the bone with those openings of the medullary nail, through which the locking pins need to be put in as well. This noticeably simplifies the insertion of the locking screws into bone and medullary nail.

In some exemplary embodiments of the medullary nail according to the invention it also possesses at least one opening or a guide wire passage for a guide wire beyond the uptake means, which guides the guide wire inside the medullary nail at least in the region of the opening/the passage. To this end, the guide wire can be guided relatively tightly through the opening or this opening can be just slightly wider than the diameter of the guide wire. This permits an effective guiding of the guide wire in the region of the opening using the guide wire passage and relative to the medullary nail.

In certain exemplary embodiments of the medullary nail according to the invention, the guide wire opening(s) of the uptake means is/are not provisioned in a centre point of symmetry of the uptake means. In this way, an alignment of the uptake means as described above is possible, advantageously, even if the uptake means is, for example, a sphere.

In some exemplary embodiments of the medullary nail according to the invention, the passage openings for the locking screw(s) are penetrations, in the region of which the outside surface of the medullary nail is disrupted or opened in relation to an outside of the medullary nail.

In some exemplary embodiments of the medullary nail according to the invention, the at least one uptake means is not an integral part of the medullary nail, but a component separate thereof, which is, where applicable, exchangeable.

In some exemplary embodiments of the medullary nail according to the invention, the uptake means is alignably or movably or shiftably or pivotably disposed and/or mounted in the shaft and/or on the shaft in the region of at least one opening of the medullary nail in at least one spatial direction—for example in a direction perpendicular to the longitudinal axis of the bone or medullary nail shaft—and/or in a plane, particularly in a plane containing the direction described previously.

The uptake means possesses in some exemplary embodiments of the medullary nail according to the invention an opening or a borehole for the uptake of a locking screw or several such or other locking means. After the insertion of the locking screw(s) into the shaft, this uptake means itself can be locked, blocked or arrested, respectively.

In some exemplary embodiments of the medullary nail according to the invention, one, several or all of the uptake means present are implemented as sleeves or at least possess such a sleeve.

Herein applies that when a sleeve is mentioned below, this term is not to be understood as limited to that. That which is said on the subject of the sleeve is also true in other exemplary embodiments according to the invention for the uptake means in its widest sense, as long as the person skilled in the art would not see a contradiction therein.

In some exemplary embodiments of the medullary nail according to the invention, the pivotability of the sleeve and its non-pivotability after the locking or blocking can be determined or demonstrated with a permitted or a prevented movement, particularly tiltability or rotatability, with a virtual axis or straight line, which can be defined in relation to the sleeve. This axis can be a longitudinal axis or an axis of symmetry of the opening or the borehole of the sleeve.

In some exemplary embodiments of the medullary nail according to the invention, the uptake means can be a means with at least one prefabricated passage opening for the locking screw. According to the invention, the term “sleeve” or uptake means comprises in addition to a structure, which the person skilled in the art would recognize as a sleeve or uptake means, any means, which do not possess a prefabricated passage opening, but in which a passage for the locking screw is created, when a locking screw is inserted, or, where applicable, the passage is only created as a result of the insertion.

In some exemplary embodiments of the medullary nail according to the invention, the term “sleeve” or uptake means thus comprises a mass, particularly a curable mass, like bone cement, which allows at an early, first point in time an alignment of the locking screw(s) guided through, but not at a later, second point in time. An arrest using a material, which takes different shapes at different environmental temperatures and which can expand or contract in the heat, is comprised according to the invention by the term uptake means or sleeve in some exemplary embodiments, if the effect of the arrest is caused or promoted through these material properties.

In some exemplary embodiments of the medullary nail according to the invention, the sleeve is a drum with a drum axis, which is, for example, perpendicular to the longitudinal axis of the shaft of the locking nail. In other exemplary embodiments of the medullary nail according to the invention, it is advantageously spherical or of elliptical or ellipsoidal diameter, such that it is freely pivotably mounted in several, preferably in all, spatial axes in the shaft.

In some exemplary embodiments of the medullary nail according to the invention, the uptake means is composed of several parts and/or materials (composite). For example, a plastic ring can be employed as part of the uptake means. Advantageously, it can prevent an unintentional unscrewing of the locking screw(s).

In some exemplary embodiments of the medullary nail according to the invention, it possesses at least or exactly one clamping means, in particular coaxially located with respect to the shaft of the medullary nail and/or essentially parallel with respect to the longitudinal axis of the medullary nail, for example a clamping screw for preventing the alignability of the at least one or of several or all uptake means and/or for fixing of their position.

According to the invention, under the “borehole”, which is provisioned in the uptake means, a borehole is understood as it is known to the person skilled in the art. Additionally a passage opening can be provisioned according to the invention for the uptake of the locking screw or of a section thereof, which the person skilled in the art would not recognize as a borehole. Such a passage opening can for example be moulded or can be a (by-)product of a moulding process. In a moulding process, unlike when a borehole is created, no sleeve material needs to be ablated or removed. Such a non-drilled passage opening can furthermore be created by bringing together several partial uptake means with according recesses.

In some exemplary embodiments of the medullary nail according to the invention, a locking signifies the prevention of a movement, for example of a medullary nail, in a longitudinal axis thereof or around its longitudinal axis, after the completion of its insertion into the bone. In connection with the present invention, it also signifies a fixation of fragments of a fracture or other bone segments. Fixation also denotes the attempt to preserve the distance between bone segments or fragments. A sintering, particularly a medically desired sintering, of the bone segments, in which the distance may change to a limited extent despite locking, is not detrimental to the fixation herein. Therefore, a locking screw or locking means in the sense of the present invention can be a means for fixing of the medullary nail in the bone as well as a means of securing at least two bones or bone segments or bone fragments relative to each other. By using a medullary nail constructed according to the invention, in some exemplary embodiments, minor misalignments of the locking screw can be compensated virtually automatically, when a locking screw is inserted into the medullary nail. In addition, the operator has the possibility to, still during operation, especially with a spherical sleeve, which can be aligned in three degrees of freedom, vary the angle of the locking screw(s) penetrating the medullary nail, for example with respect to the shaft, and/or the spatial alignment of the locking screw(s). Thereby, it is possible for the operator to precisely reposition, to adapt anatomically in the correct way or to compress, respectively, the fracture fragments of the bone or of the joint.

The locking of the uptake means with the locking screw or the fixing of the position or the blocking of the uptake means, respectively, can be achieved in several ways. The uptake means, which is made of plastic only in a preferred embodiment and which, independently thereof, can be, similarly to known wall plugs, equipped with a slot, can be widened and fixed for example through a conical or other embodiment of the locking screw. Equally, it is possible to achieve the fixation of the position of the uptake means with the locking screw by using a clamping screw, which is arranged for example coaxially and/or essentially parallel to the shaft of the medullary nail.

Such a clamping screw may be implemented for example as a dowel pin or a lockable means or any other means or be replaced by such, which can exert a pushing or pulling effect on the uptake means for its immobilisation in space or at least in one plane. If several uptake means are provisioned in the medullary nail, these can be fixed, particularly fixed with respect to position, angle and/or orientation, for example by intermediary bodies being provisioned between the individual uptake means, wherein the individual uptake means are then fixed through a means, for example a clamping screw. Combinations of the aforementioned and further means known to the person skilled in the art of the kind mentioned above for achieving an arrest or fixation are comprised by the invention as well.

However, such a means—like an intermediary body—is not indispensable. Rather, the uptake means can alternatively be interlocked with each other while there is direct contact of the of the uptake means with each other.

The intermediary bodies can be designed as a solid part or with at least one hollow cavity. The hollow cavities can result in a greater deformity of the intermediary bodies, which in turn increases the friction and, thus, the angular stability.

The intermediary bodies can be designed such that they can positively connect with the uptake means, similarly to a gearing, in order to offer an increased frictional resistance in the interlocking and thus increase the angular stability.

The medullary nail according to the invention can be designed in a modular setup, such that the medullary nail contains a coaxial borehole or opening, into which an insert with at least one uptake means or a contiguous unit of uptake means and intermediary bodies can be inserted. Advantageously, this embodiment allows that before the insertion of the insert the opening is usable for a cannulation, with the advantages in relation to cannulation known to the person skilled in the art.

Uptake means can be filled with placeholders instead of locking screws, too. This offers the advantage that an uptake means not filled with a locking screw permits a desired flux of forces through itself using a placeholder, if after the completion of the insertion of the medullary nail into the bone the axial tension is utilised for an angularly stable securement.

It is pointed out that the medullary nail according to the invention can be used together with one or several locking screws. Thus the term “locking screw” and “locking screws” is used interchangeably in the present description: where a “locking screw” is mentioned, “locking screws” are meant as well and vice versa, wherever this is technically possible. Therein, the term “locking screw” stands for a locking means in general.

Thus, the locking screw can be implemented as a locking pin. A screw thread is not required according to the invention. Advantageously, the employment of a fit or other means for increasing the friction between the uptake means and the locking screw can prevent that the locking screw moves out of the uptake means along its longitudinal axis undesirably over time. These means for increasing friction can be provisioned in only one section (or along one section) of the uptake means and/or the locking screw or in its entire length.

The term “preventing” describes in some exemplary embodiments of the medullary nail according to the invention a termination of the alignability of the locking means inserted into the uptake means, for example of a locking screw, in space, at least in at least one spatial direction or one rotational direction. This can occur through a blocking, arresting or curing, a locking or suchlike of the uptake means. To this end, a preventing means such as a clamping screw as mentioned above can be employed. Preventing, however, can also mean an effect or property, which is inherent to one of the structures. An example would be the curability of an uptake means designed using cement.

A further example how the alignability of the uptake means can be prevented according to the invention is the provision of a means for increasing the friction between locking means and bone nail, wherein an insertion of the locking means (for example using screwing, driving in, insertion or suchlike) into the uptake means increasingly or abruptly terminates the alignability of the uptake means. This can be achieved by spreading apart the uptake means using the for example tapered shaft of the locking means. By such spreading apart or just by an according increase of pressure with a deformation of an outer contour of the uptake means or at least by an increase of pressure of the uptake means onto its surroundings, the uptake means can be pressed against a further structure of the medullary nail in such a way that the uptake means is blocked in relation to the medullary nail in its freedom of movement and in particular in its alignability. It can thus be arrested by just driving the locking means into itself.

The locking means (for example in the shape of a pin or screw) can be designed such that only the insertion of its section to be inserted last into the uptake means causes the blocking. The locking means can be prepared for such a blocking. The uptake means can be designed accordingly as well. In addition, the locking means as well as the uptake means can be designed for achieving the blocking or locking, respectively.

All items according to the invention can be made of a type II anodised titanium alloy (Ti6A14V) for better biomechanical and biomedical performance.

The invention is illustrated in the following, using figures. In these:

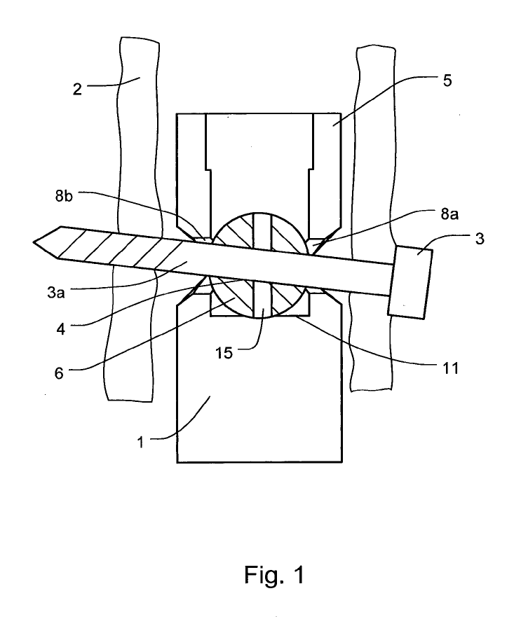

FIG. 1 shows a medullary nail according to the invention with a sleeve pivotably arranged inside the shaft of the medullary nail as an example for an uptake means, which takes up a locking screw for the fixation of the medullary nail;

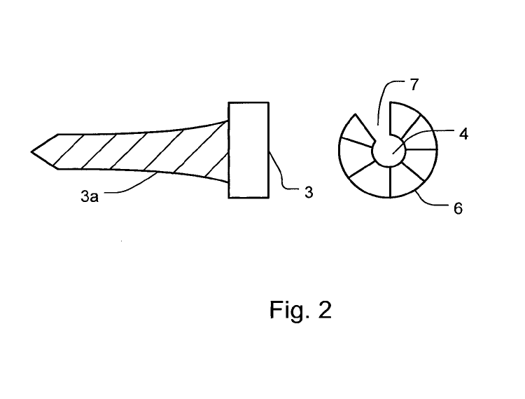

FIG. 2 shows a schematic representation of a locking screw and a sleeve for locking the medullary nail in a bone;

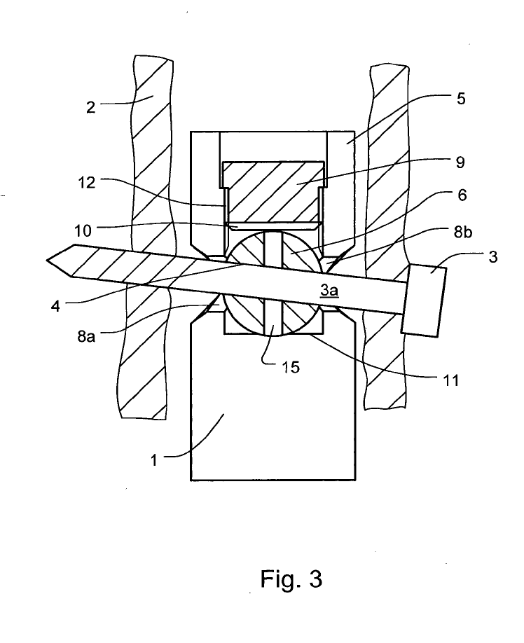

FIG. 3 shows a medullary nail implanted into the medullary cavity of a bone with a clamping screw for the positionally stable fixation of the medullary nail; and

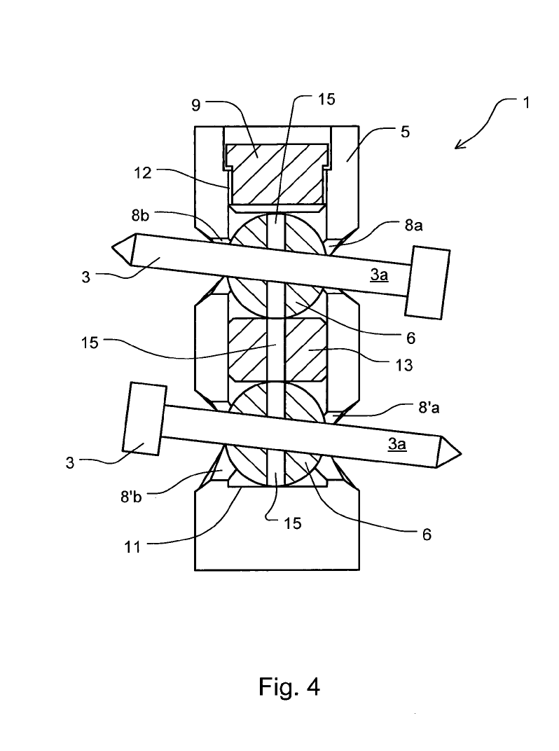

FIG. 4 shows a cross section through a medullary nail with two uptake means, each designed for the uptake of a locking screw, which are fixed to each other by an intermediary body and a clamping screw.

FIG. 1 shows a medullary nail 1, which is implanted inside the medullary cavity of a bone 2. Inside the medullary nail 1, in the region, in which a locking screw 3 is to be placed, a spherical sleeve 6 is inserted into a shaft 5 of the medullary nail 1 as an example for an uptake means, which contains a borehole 4 for taking up the locking screw 3. The shaft 5 contains in the region of the sleeve 6 two opposite penetrations 8a, 8b, through which the locking screw 3 can be guided, wherein the section 3a comes to rest inside the sleeve 6. The sleeve 6 rests on a seating surface 11 in the medullary nail 1 and is mounted freely rotatably in the shaft 5. Thereby, the locking screw 3 can be driven through the borehole 4 in the medullary nail 1 during the operation in a range of angles limited by the penetrations 8a, 8b in the shaft in any inclination. Also, small misalignments of the sleeve 6 or of the locking screw 3 are compensated, such that the locking screw 3 always engages with the borehole 4 of the sleeve 6.

Due to this angular flexibility or angular alignability, respectively, when inserting the locking screw 3, it is possible to optimally adapt the medullary nail 1 to the individual anatomical or pathological, respectively, situation of the patient during the implantation.

The locking of the sleeve 6 with the shaft 5 can be accomplished in different ways.

The sleeve 6 of the medullary nail 1 possesses as an example for an uptake means 6 a guide wire opening 15 penetrating therethrough for taking up a guide wire not shown in the figures.

One or several such guide wire openings 15 can also be present in each of the uptake means or sleeves discussed below or shown in the figures, but also in intermediary bodies or support bodies 13, as shown in the following figures.

One example for an uptake means with a locking screw according to the invention is shown in FIG. 2. The locking screw 3 herein contains a partially or completely conical screw shaft 3a; the sleeve 6 with its opening 4 is provisioned with a slot 7, similar to a wall plug. The sleeve 6 is then widened when screwing in the locking screw 3 and is spread apart in the shaft 5 of the medullary nail 1. The sleeve then lays itself onto the according stops, for example onto the edges of the penetrations 8a and 8b. Through this positive fixation of the spherical sleeve 6 inside the shaft 5, the locking screw 3 is locked into the chosen position permanently in an angularly stable way.

In order to achieve an optimum fixation, the sleeve 6 can be made of or contain such a spreadable material, for example plastic suitable for this application.

In FIG. 3, another possibility for an angularly stable arrest of the sleeve 6 with the locking screw 3 is shown. The design of the medullary nail 1, the sleeve 6 and the locking screw 3 is similar to those shown in FIG. 1. Herein, the spherical sleeve 6 can consist of an according plastic material again. The sphere or sleeve 6, respectively, rests inside the shaft 5 of the medullary nail on a seating surface 11.

Above the sphere or sleeve 6, respectively, the shaft 5 is provided with a screw thread 12, into which a clamping screw 9 is inserted coaxially to the shaft, which rests on the top surface of the sleeve 6. By screwing the clamping screw 9 into the screw thread 12, the sleeve is pressed onto the seating surface 11 and is fixed positionally stably after the insertion of the locking screw 3.

In order to increase the clamping force and the resulting loosening torque of the sleeve 6 in the shaft 5, the clamping screw 9 can be adapted accordingly geometrically to the shape of the sleeve at its contact surface 10 with the sleeve 6, which is not shown here. Thereby, the static friction between the clamping screw and the sleeve can be increased.

In addition, the contact surface of the clamping screw can be roughened, for example by knurling and the application of coatings which increase friction. Another possibility would be to provide the clamping screw 9 with a tapered, pointed end, which penetrates into the sleeve 6 and thereby additionally fixes it positively.

In FIG. 4, a medullary nail 1 is shown with a shaft 5, which possesses several, in this case two, penetrations 8a, 8b and 8′a, 8′b, wherein in these two regions, one sleeve 6 is inserted each, through each of which one locking screw 3 can be driven.

For locking the two sleeves 6, a clamping screw 9 is provisioned according to FIG. 3, which contacts the upper sleeve 6. Between the two sleeves 6, a block shaped intermediary body 13 is provisioned, which contacts both sleeves 6. By tightening the clamping screw 9, both sleeves 6 are then arrested positionally stably, with the interposition of the intermediary body 13. The intermediary body 13 also possesses (at least) one guide wire opening 15.

1. Medullary nail (1), for the insertion into a long bone, with a shaft (5) with a longitudinal axis, wherein the shaft possesses at least one passage opening (8) for inserting at least one locking means, in particular one locking screw (3) or one locking pin, into an inside of the medullary nail (1) and/or for guiding through the medullary nail (1);
wherein the medullary nail (1) possesses at least one uptake means (6) for taking up at least one section (3a) of the locking means;
wherein the at least one uptake means (6) is disposed inside the shaft (5) or inside the passage opening (8) while it is in at least one of its spatial axes movable or alignable, particularly rotatable or pivotable, in relation to the longitudinal axis of the shaft (5);
wherein the medullary nail (1) possesses at least one means, particularly an arresting means (9) and/or an embodiment of locking means and/or uptake means (6), for preventing the alignability of the at least one uptake means (6) in relation to the longitudinal axis and/or to a transverse axis perpendicular thereto; and wherein
the at least one uptake means (6) possesses at least one guide wire opening (15) passing therethrough for taking up a guide wire.

2. Medullary nail according to claim 1, characterised in that the uptake means (6) is a sleeve (6) or a drum with a sleeve or drum axis perpendicular to the longitudinal axis of the shaft (5) of the locking medullary nail (1).

3. Medullary nail according to claim 1, characterised in that the uptake means (6) is in at least one section thereof spherical and is freely pivotably or rotatably arranged, in particular mounted, in more than one spatial axis, particularly in all spatial axes, in the shaft (5).

4. Medullary nail according to claim 1, characterised by a means for the non-positive and/or positive arresting of the uptake means (6) through a non-positive connection while preventing the alignability.

5. Medullary nail according to claim 1, characterised in that the uptake means (6) is slotted or widenable and that the associated locking screw (3) is designed conically at least in one section for positional fixation.

6. Medullary nail according to claim 1, characterised by at least or exactly one clamping screw (9) for preventing the alignability of the at least one uptake means (6) and/or for its positional fixation.

7. Medullary nail according to claim 1, characterised in that the uptake means (6) contains plastic material or consists of plastic material.

8. Medullary nail according to claim 1, characterised in that in several sequential uptake means (6) inside or on the medullary nail (1) support bodies (13) contacting the uptake means (6) are provisioned between the uptake means (6), wherein the arrangement of the uptake means (6) and support bodies (13) is compressible and/or blockable and/or arrestable by at least one clamping screw (9), which acts along the longitudinal direction of the medullary nail (1).

9. Medullary nail according to claim 1, characterised in that at least one of the support bodies (13) possesses at least one guide wire opening (15).

10. Medullary nail according to claim 1, characterised in that in several sequential uptake means (6) in or on the medullary nail (1) at least two of the uptake means (6) compress or block or arrest each other through the influence of a clamping screw (9).

11. Medullary nail according to claim 2, characterised by a means for the non-positive and/or positive arresting of the uptake means (6) through a non-positive connection while preventing the alignability.

12. Medullary nail according to claim 2, characterised in that the uptake means (6) is slotted or widenable and that the associated locking screw (3) is designed conically at least in one section for positional fixation.

13. Medullary nail according to claim 2, characterised by at least or exactly one clamping screw (9) for preventing the alignability of the at least one uptake means (6) and/or for its positional fixation.

14. Medullary nail according to claim 2, characterised in that the uptake means (6) contains plastic material or consists of plastic material.

15. Medullary nail according to claim 2, characterised in that in several sequential uptake means (6) inside or on the medullary nail (1) support bodies (13) contacting the uptake means (6) are provisioned between the uptake means (6), wherein the arrangement of the uptake means (6) and support bodies (13) is compressible and/or blockable and/or arrestable by at least one clamping screw (9), which acts along the longitudinal direction of the medullary nail (1).

16. Medullary nail according to claim 2, characterised in that at least one of the support bodies (13) possesses at least one guide wire opening (15).

17. Medullary nail according to claim 2, characterised in that in several sequential uptake means (6) in or on the medullary nail (1) at least two of the uptake means (6) compress or block or arrest each other through the influence of a clamping screw (9).
